(19) United States
(12) Patent Application Publication
(10) Pub. No.: US 2004/0132037 A1
(43) Pub. Date: Jul. 8, 2004

(54) Materials and methods for the induction of premature chromosone condensation

(76) Inventors: Pataje G S Prasanna, Gaithersburg, MD (US); William F Blakely, Silver Spring, MD (US)

Correspondence Address: MORGAN LEWIS & BOCKIUS LLP, 1111 PENNSYLVANIA AVENUE NW, WASHINGTON, DC, 20004, US

(21) Appl. No.: 10/469,075

(22) PCT Filed: Feb. 28, 2002
(86) PCT No.: PCT/US02/05752

(51) Int. Cl. C12Q 1/68; C12N 15/85

(52) U.S. Cl. 435/6; 435/455

(57) ABSTRACT

The present invention provides a simple and rapid method to study chromosome aberrations using unstimulated cells. Premature chromosome condensation is induced by incubating unstimulated cells in the presence of a mitosis-enhancing factor. The condensed chromosomes produced by the present methods can be used in numerous forms of cytogenetic analysis, in particular, with in situ hybridization probes and chromosome painting. This technique can be applied to biological dosimetry of radiation exposures involving uniform whole-body low-linear energy transfer exposures.

### FIELD OF THE INVENTION

The present invention concerns the fields of cytogenetics, molecular cytogenetic, cell biology, genetic toxicology and genomics. In particular, the present invention concerns methods of inducing premature chromosome condensation and methods of analyzing genetic material using the condensed chromosomes.

### BACKGROUND

Various environmental insults have the potential to induce physical damage to genetic material. In addition to exposure to environment toxins, accidental exposure of human beings to radiation is a major concern. Development of simple and rapid methods is required for insult dose assessment, which will benefit the treatment of exposed individuals.

Muller and Streffer (Muller et al. (1991) Int. J. Radiat. Biol. 59, 863-873) published a comprehensive review of biological indicators of radiation damage, explaining current techniques of biological dosimetry for radiation dose assessment. After exposure to high doses of radiation, sufficient numbers of mitotic cells are not available for dose assessment by the routine metaphase spread chromosome aberration analysis. The premature chromosome condensation (PCC) assay, performed on an exposed individual's blood lymphocytes, is viewed as a rapid biodosimetry method of clinical significance (Pantelias et al. (1985) Mutat. Res. 149, 67-72; Blakely et al. (1995) Stem Cells 13, 223-230; and Prasanna et al. (1997) Health Phys. 72, 594-600.

Currently, physical damage to chromosomes can be analyzed by observation of chromosomes after preparation of a metaphase spread. Chromosomes are visualized in mitotic cells following a short-term cell culture in which cells are stimulated into proliferation by a mitogen and then subjected to cell cycle arrest with colchicine or colcemid. The chromosomes are observed under a microscope after being treated either by staining or by hybridizing with a fluorescent probe. This technique depends upon the successful stimulation of the cells to proliferate and requires 48 hours or more of cell culture to obtain useful yields. The technique is labor intensive and requires experience in cytogenetic techniques to practice. The analysis is further complicated by cell killing and cell cycle delay induced by the treatment. In addition, the low yield of condensed chromosomes frequently requires large numbers of metaphase spreads to obtain statistically significant data.

Another method of analyzing physical damage to chromosomes involves inducing the premature chromosome condensation (PCC) in the cells and preparing a chromosome spread. Historically, premature chromosome condensation was accomplished by fusing the cells of interest with mitotic cells. This resulted in the condensation of the chromosomes in the test cells into chromatid-like structures. Although this technique does produce premature chromosome condensation, there are several difficulties associated with its practice. The technique requires a constant supply of mitotic cells to be fused with the test cells. The culture and maintenance of the mitotic cells adds considerably to the expense of the method. Additionally, cell fusion techniques (for example, PEG mediated fusion) are inefficient and produce low and variable yields of fused cells. This results in a low and variable yield of premature chromosome condensation in the test cells (Pantelias et al. (1983) Somatic Cell Genet. 9, 533-547).

The deficiencies of mitotic cell fusion to induce premature chromosome condensation are well known in the art and the search for alternative simple and rapid protocols has been a topic of ongoing research (Gotoh et al. (1996) Int. J. Radiat. Biol. 70, 517-520; Kanda et al. (1999) Int. J. Radiat. Biol. 75, 441-446; Durante et al. (1998) Int. J. Radiat. Biol. 74, 457-462; and Coco-Martin et al. (1997) Int. J. Radiat. Biol. 71, 265-273). Recently, premature chromosome condensation has been induced by stimulating cells with a mitogen and then culturing the cells in the presence of phosphatase inhibitors. Inhibitors of type 1 and 2A protein phosphatases have been used to induce PCC in proliferating cells (Gotoh et al. (1996) Int. J. Radiat. Biol. 70, 517-520; Kanda et al. (1999) Int. J. Radiat. Biol. 75, 441-446; Durante et al. (1998) Int. J. Radiat. Biol. 74, 457-462; and Coco-Martin et al. (1997) Int. J. Radiat. Biol. 71, 265-273).

The condensed chromosomes prepared by phosphatase inhibitor treatment were evaluated for biological dosimetry applications using chromosome aberration analysis in PCC spreads. Premature chromosome condensation was induced by okadaic acid (OA) (Gotoh et al. (1996) Int. J. Radiat. Biol. 70, 517-520; Kanda et al. (1999) Int. J. Radiat. Biol. 75, 441-446) or calyculin A (Durante et al. (1998) Int. J. Radiat. Biol. 74, 457-462) in mitogen stimulated cells and obtained 48 hours after mitogen-stimulation. Durante et al. (Durante et al. (1998) Int. J. Radiat. Biol. 74, 457-462) demonstrated that simultaneous measurement of chromosome aberrations in G₁ and M phases is possible by using whole-chromosome probe fluorescence in situ hybridization (FISH) technique following exposure to 200-kVp x-rays. It has also been shown that incubation of actively dividing tumor cell lines in a cell culture medium containing OA or calyculin A results in PCC induction (Coco-Martin et al. (1997) Int. J. Radiat. Biol. 71, 265-273). Using whole-chromosome-specific probes, chemically induced PCC spreads containing radiation-induced chromosome aberrations are readily identified as cells with more than 2 chromosome spots. A difference in radiosensitivity was demonstrated between radiosensitive (SCC61) and radioresistant (A549) cell lines (Coco-Martin et al. (1997) Int. J. Radiat. Biol. 71, 265-273).

Although the use of phosphatase inhibitors produces premature chromosome condensation in stimulated or proliferating cells, presently available methods still require an incubation period in order to produce sufficiently high yields of premature chromosome condensation to be useful for chromosome aberration analysis.

### BRIEF SUMMARY OF THE INVENTION

Notwithstanding the methods discussed above, there exists a need in the art for rapid and simple methods to assess the damage of genetic material by environmental insults. Presently, a major cause of the difficulty in making such assessments is the time and labor required to generate condensed chromosomes for subsequent analysis. The present invention meets this long felt need by providing a cell culture medium that induces premature chromosome condensation rapidly and in high yields in unstimulated cells. The present invention does away with the need for cell fusion to induce premature chromosome condensation in unstimulated cells and does away with the need for stimulation and subsequent incubation required by other methods known in the art. Condensed chromosomes prepared using the materials and methods of the present invention have been used to demonstrate that damage to specific chromosomes in unstimulated HPBL can be studied by FISH with whole-chromosome-specific probes in chemically-induced PCC spreads. The methods of the present invention are simpler and faster than those known in the art and are particularly suited to automated, high throughput assays of chromosome damage. These methods have numerous applications including rapid biological dosimetry applications.

The present invention provides a cell culture medium for inducing premature chromosome condensation in a cell. In preferred embodiments, the cell culture medium comprises one or more mitosis enhancing factors. In some embodiments, the mitosis enhancing factor may be one or more cyclins, cyclin kinases, histone kinases, cyclins, topoisomerases, structural maintenance of chromosome (SMC) proteins, histones, cdk1 substrate, and components of mitosis promoting factor. In a preferred embodiment, the mitosis enhancing factor is p34^{cdc2}/cyclin B kinase.

A cell culture medium of the present invention may comprise a phosphatase inhibitor. In such cases, the phosphatase inhibitor may include one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. A cell culture medium of the invention may comprise an energy source, preferably ATP and/or GTP.

The present invention provides a method of analyzing a chromosome by incubating a cell with a medium comprising a mitosis enhancing factor, wherein the medium induces premature chromosome condensation, and analyzing the condensed chromosome. In some embodiments, the mitosis enhancing factor may be one or more of cyclin kinases, histone kinases, cyclins, topoisomerases, SMC proteins, cdk1 substrate, histones, and components of mitosis promoting factor (MPF). In some preferred embodiments, the mitosis enhancing factor may include p34^{cdc2}/cyclin B kinase.

A medium for use in the method of analyzing a chromosome may comprise a phosphatase inhibitor. Preferably, the phosphatase inhibitor may be one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. The medium may comprise an energy source, preferably, ATP and/or GTP. The medium may include a transfection reagent.

The method for analyzing a chromosome may be practiced on any type of cell. In some embodiments, the cell may be a lymphocyte. Preferably, the cell is a mammalian cell. In some embodiments, the cell is a human peripheral blood lymphocyte. In some embodiments, the cell is a murine cell, preferably a murine peripheral blood lymphocyte.

The method of analyzing a chromosome may include preparing a chromosome spread. The method may include hybridizing one or more oligonucleotides to one or more chromosomes and enumerating chromosome spots. In some embodiments, one or more of the oligonucleotides comprises a detectable moiety. Preferably, the detectable moiety is a fluorescent moiety although the detectable moiety may be one or more of biotin, digoxigenin, antigens, enzymes and haptens.

The present invention also provides a method of assessing clastogenicity of a compound by contacting a cell with the compound, incubating the cell with a medium comprising a mitosis enhancing factor, wherein the medium induces premature chromosome condensation and analyzing the condensed chromosomes for breakage, structural and/or numerical aberrations. In some embodiments, the cell is contacted with the medium and the compound simultaneously. In other embodiments, the cell may be contacted with the compound and then transferred to a suitable medium. It may be desirable in some instances to incubate the cell after contact with the compound for a period of time sufficient to allow chromosomal repair. In some embodiments, the mitosis enhancing factor may be one or more of cyclin kinases, histone kinases, cyclins, topoisomerases, SMC proteins, cdk1 substrate, histones, and components of mitosis promoting factor (MPF). In some preferred embodiments, the mitosis enhancing factor may include p34^{cdc2}/cyclin B kinase.

A medium for use in the method of assessing clastogenicity of a compound may comprise a phosphatase inhibitor. Preferably, the phosphatase inhibitor may be one or more of okadaic acid, salts of okadaic acid, calyculin A, cantharidic acid, cantharidin, cypermethrin, deltamethrin, dephostatin, 3,4dephostatin, endothall, fenvalerate, fostriecin, microcystin-LA, microcystin-LF, microcystin-LR, microcystin-LW, microcyctin-RR, and microcystin-YR. The medium may comprise an energy source, preferably, ATP and/or GTP. The medium may include a transfection reagent.

The method of assessing clastogenicity of a compound may be practiced on any type of cell. In some embodiments, the cell may be a lymphocyte. Preferably, the cell is a mammalian cell. In some embodiments, the cell is a human peripheral blood lymphocyte. In some embodiments, the cell is a murine cell, preferably a murine peripheral blood lymphocyte.

The method of assessing clastogenicity of a compound may include preparing a chromosome spread. The method may include hybridizing one or more oligonucleotides to one or more chromosomes and enumerating chromosome spots. In some embodiments, one or more of the oligonucleotides comprises a detectable moiety. Preferably, the detectable moiety is a fluorescent moiety although the detectable moiety may be one or more of biotin, digoxigenin, antigens, enzymes and haptens.

The present invention also provides a method of assessing toxicity of a compound by contacting a cell with the compound, incubating the cell with a medium comprising a mitosis enhancing factor, wherein the medium induces premature chromosome condensation and analyzing the condensed chromosomes. In some embodiments, the cell is contacted with the medium and the compound simultaneously. In other embodiments, the cell may be contacted with the compound and then transferred to a suitable medium. It may be desirable in some instances to incubate the cell after contact with the compound for a period of time sufficient to allow chromosomal repair. In some embodiments, the mitosis enhancing factor may be one or more of cyclin kinases, histone kinases, cyclins, topoisomerases, SMC proteins, cdk1 substrate, histones, and components of mitosis promoting factor (MPF). In some preferred embodiments, the mitosis enhancing factor may include p34^{cdc2}/cyclin B kinase.

A medium for use in the method of assessing toxicity of a compound may comprise a phosphatase inhibitor. Preferably, the phosphatase inhibitor may be one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. The medium may comprise an energy source, preferably, ATP and/or GTP. The medium may include a transfection reagent.

The method of assessing toxicity of a compound may be practiced on any type of cell. In some embodiments, the cell may be a lymphocyte. Preferably, the cell is a mammalian cell. In some embodiments, the cell is a human peripheral blood lymphocyte. In some embodiments, the cell is a murine cell, preferably a murine peripheral blood lymphocyte.

The method of assessing toxicity of a compound may include preparing a chromosome spread. The method may include hybridizing one or more oligonucleotides to one or more chromosomes and enumerating chromosome spots. In some embodiments, one or more of the oligonucleotides comprises a detectable moiety. Preferably, the detectable moiety is a fluorescent moiety although the detectable moiety may be one or more of biotin, digoxigenin, antigens, enzymes and haptens.

The present invention also provides a method of detecting chromosomal abnormalities in a subject by isolating one or more cells from the subject, incubating the cell with a medium comprising a mitosis enhancing factor, wherein the medium induces premature chromosome condensation and analyzing the condensed chromosomes for abnormalities. In some embodiments, the mitosis enhancing factor may be one or more of cyclin kinases, histone kinases, cyclins, topoisomerases, SMC proteins, cdk1 substrate, histones, and components of mitosis promoting factor (MPF). In some preferred embodiments, the mitosis enhancing factor may include p34^{cdc2}/cyclin B kinase.

A medium for use in the method of detecting chromosomal abnormalities in a subject may comprise a phosphatase inhibitor. Preferably, the phosphatase inhibitor may be one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. The medium may comprise an energy source, preferably, ATP and/or GTP. The medium may include a transfection reagent.

The method of detecting chromosomal abnormalities in a subject may be practiced on any type of cell. In some embodiments, the cell may be a lymphocyte. Preferably, the cell is a mammalian cell. In some embodiments, the cell is a human peripheral blood lymphocyte. In some embodiments, the cell is a murine cell, preferably a murine peripheral blood lymphocyte. In some embodiments, the cell may be obtained from a subject while the subject is in utero.

The method of detecting chromosomal abnormalities in a subject may include preparing a chromosome spread. The method may include hybridizing one or more oligonucleotides to one or more chromosomes and enumerating chromosome spots. In some embodiments, one or more of the oligonucleotides comprises a detectable moiety. Preferably, the detectable moiety is a fluorescent moiety although the detectable moiety may be one or more of biotin, digoxigenin, antigens, enzymes and haptens.

The present invention also provides a method of assessing a radiation dose received by a subject by isolating one or more cells from the subject, contacting one or more cells with a medium comprising a mitosis enhancing factor, wherein the medium induces premature chromosome condensation and analyzing the condensed chromosomes for abnormalities such as breakage, structural and/or numerical aberrations. In some embodiments, the mitosis enhancing factor may be one or more of cyclin kinases, histone kinases, cyclins, topoisomerases, SMC proteins, cdk1 substrate, histones, and components of mitosis promoting factor (MPF). In some preferred embodiments, the mitosis enhancing factor may include p34^{cdc2}/cyclin B kinase.

A medium for use in the method of assessing a radiation dose received by a subject may comprise a phosphatase inhibitor. Preferably, the phosphatase inhibitor may be one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. The medium may comprise an energy source, preferably, ATP and/or GTP. The medium may include a transfection reagent.

The method of assessing a radiation dose received by a subject may be practiced on any type of cell. In some embodiments, the cell may be a lymphocyte. Preferably, the cell is a mammalian cell. In some embodiments, the cell is a human peripheral blood lymphocyte. In some embodiments, the cell is a murine cell, preferably a murine peripheral blood lymphocyte.

The method of assessing a radiation dose received by a subject may include preparing a chromosome spread. The method may include hybridizing one or more oligonucleotides to one or more chromosomes and enumerating chromosome spots. In some embodiments, one or more of the oligonucleotides comprises a detectable moiety. Preferably, the detectable moiety is a fluorescent moiety although the detectable moiety may be one or more of biotin, digoxigenin, antigens, enzymes and haptens.

The present invention also provides a composition comprising a cell and a cell culture medium, wherein the cell culture medium comprises a mitosis enhancing factor and induces premature chromosome condensation in the cell. In the compositions of the present invention, the mitosis enhancing may be one or more of cyclin kinases, histone kinases, cyclins, topoisomerases, structural maintenance of chromosome (SMC) proteins, histones, cdk1 substrate, and components of mitosis promoting factor. In some preferred embodiments, the mitosis enhancing factor may be p34^{cdc2}/cyclin B kinase. The compositions of the present invention may include a phosphatase inhibitor. The phosphatase inhibitor may be one or more of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR. The compositions may also comprise an energy source, preferably ATP and/or GTP.

The present invention provides kits for the induction of premature chromosome condensation in test cells. In some embodiments, the kits may comprise one or more containers of a cell culture medium which comprises a mitosis enhancing factor and induces premature chromosome condensation in the cell. The mitosis enhancing may be one or more of cyclin kinases, histone kinases, cyclins, topoisomerases, structural maintenance of chromosome (SMC) proteins, histones, cdk1 substrate, and components of mitosis promoting factor. In some preferred embodiments, the mitosis enhancing factor may be p34^{cdc2}/cyclin B kinase. The kits of the present invention may include one or more containers holding one or more phosphatase inhibitors. The phosphatase inhibitor may be one or more of okadaic acid, salts of okadaic acid, calyculin A, cantharidic acid, cantharidin, cypermethrin, deltamethrin, dephostatin, 3,4dephostatin, endothall, fenvalerate, fostriecin, microcystin-LA, microcystin-LF, microcystin-LR, microcystin-LW, microcyctin-RR, and microcystin-YR. The kits may also comprise one or more containers holding an energy source, preferably ATP and/or GTP. The kits of the present invention may comprise one or more containers holding one or more transfection reagents.

### BRIEF DESCRIPTION OF THE DRAWINGS

FIG. 1 is a schematic representation of the assembly and phosphorylation state of various mitosis enhancing factors in various stages of the cell cycle.

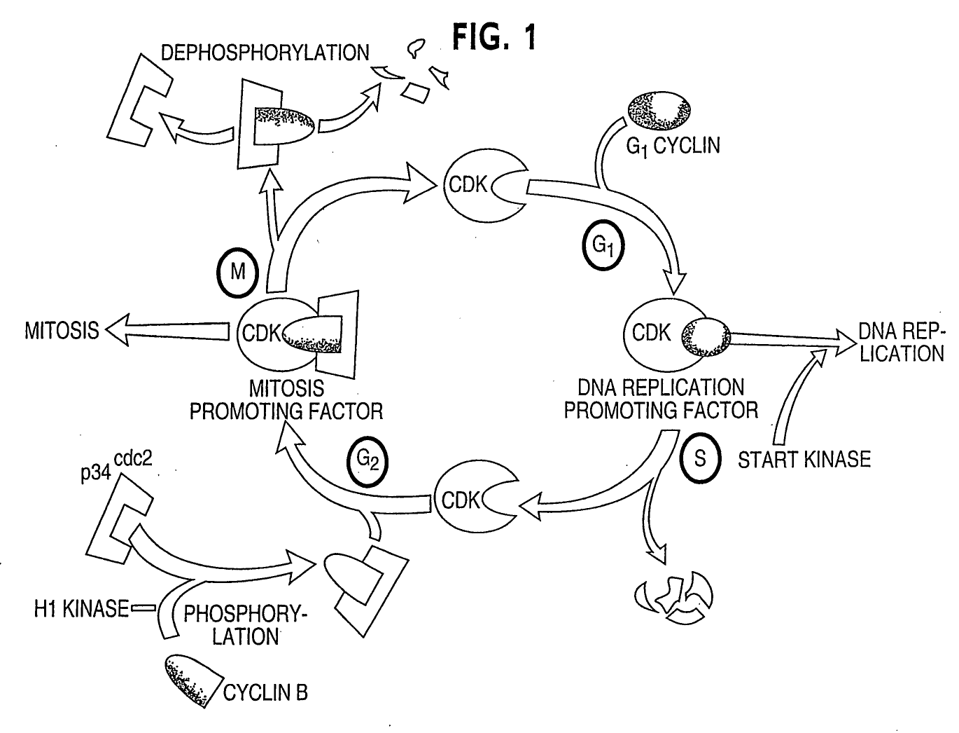

FIGS. 2A-2D show chromosome spreads of cells treated to induce premature chromosome condensation. FIG. 2A is a photomicrograph of a Giemsa stained chromosome spread of HPBLs in which premature chromosome condensation was induced by mitogen stimulation and incubation in the presence of OA. FIG. 2B is a photomicrograph of a Giemsa stained chromosome spread of HPBLs in which premature chromosome condensation was induced by incubation in the presence of p34^{cdc2}/cyclin B kinase and OA. FIG. 2C is a photomicrograph showing a FISH analysis of chromosome 1 in un-irradiated HPBLs in which premature chromosome condensation was induced by incubation in the presence of p34^{cdc2}/cyclin B kinase and OA. FIG. 2D is a photomicrograph showing a FISH analysis of chromosome 1 in irradiated HPBLs in which premature chromosome condensation was induced by incubation in the presence of p34^{cdc2}/cyclin B kinase and OA.

FIG. 3 is a graph showing the effects of various incubation times and OA concentrations on PCC index in mitogen stimulated HPBLs.

FIG. 4 is a graph showing the effects of various p34^{cdc2}/cyclin B kinase concentrations on PCC index in p34^{cdc2}/cyclin B kinase treated HPBLs.

FIG. 5 is a graph showing the dose-response curve for cells with radiation induced chromosome aberrations.

FIG. 6 is a graph showing the increase in the percentage of cells with two or more fluorescent spots in cells isolated from patients exposed to radiation when compared to normal control cells.

### DETAILED DESCRIPTION OF THE INVENTION

The present invention provides materials and methods for the induction of premature chromosome condensation in without the need to stimulate the cells with a mitogen. In addition, the present invention provides methods of analyzing genetic material by inducing premature chromosome condensation and analyzing the physical structure of the condensed chromosomes. The present invention is useful in any application requiring premature chromosome condensation in a test cell. The invention is particularly useful in the fields of cytogenetics, molecular cytogenetics, cell biology, genetic toxicology and genomics.

In some aspects, the present invention provides materials and methods useful in diagnostic cytogenetics. The materials and methods of the present invention may be used in prenatal, postnatal and pre-implantation testing to evaluate the genetic material of a test cell. For example, the methods described herein may be used to evaluate the genetic material in a potential sperm donor to determine the presence or absence of chromosomal aberrations in the sperm. Likewise, the present invention may be used to analyze the genetic material of a subject while the subject is in utero.

In some related aspects, the present invention can be used in cytogenetic research. In the field of genomics, for example, the present invention may be used to detect genes associated with various syndromes characterized by chromosomal aberrations, for example Downs syndrome. In a particular embodiment, the present invention may be used to detect genes associated with microdeletion syndromes. In another embodiment, the present invention may be used to detect chromosomal anomalies (both numerical and structural) associated with cancer. In some preferred embodiments, the present invention may be used to detect gene amplification.

In the field of environmental testing, the present invention may be used to assess the exposure of a subject to environmental insults. In some preferred embodiments, the present invention may be used to assess the radiation dose received by a subject. The radiation dose may have been received as a result of accidental exposure or may be the result of occupational exposure. The present invention may be particularly useful in cases of exposure of a large number of subjects as the capability of automating the present invention makes it well suited to a high throughput automated screening system. In other embodiments, the exposure of a subject to a compound which induces chromosomal abnormalities can be assessed.

In some preferred embodiments, the present invention provides methods of assessing the toxicity of a drug. These methods are useful in the identification of potential chemotherapeutic agents where it is desirable to have an agent capable of inducing chromosomal breaks. In this aspect, the present methods may be used to assess the clastogenicity (ability to break chromosomes) of a particular agent. The present methods may also be used as an initial safety screen to determine whether a therapeutic agent induces chromosomal aberrations.

Cells

Any type of cell having genetic material may be used in the practice of the present invention. For example, cells from heart, lung, liver, kidney, brain or other tissue may be used as a source of cells. The isolation of cells from various tissues may be accomplished using any technique known to those skilled in the art. In preferred embodiments, the cells are of mammalian origin, such as human or murine cells. In some preferred embodiments, peripheral blood lymphocytes may be used for premature chromosome condensation and analysis. In other preferred embodiments, cells may be oocytes or obtained from embryos, amniotic fluid or established cell lines, such as stem cell lines.

The isolation of the cells to be used in the present invention may be by any means known to those skilled in the art. In some preferred embodiments, human peripheral blood lymphocytes (HPBLs) may be used. The isolation of peripheral blood lymphocytes is routine in the art. One suitable protocol is described below and other methods known to those skilled in the art could be used. In the following protocol, the peripheral blood lymphocytes were isolated from a human subject. They could equivalently be isolated from any subject. In some preferred embodiments, the subject may be mammalian. In other preferred embodiments, the subject may be a human or a mouse.

Lymphocytes may be isolated from whole blood samples using any suitable technique known to those skilled in the art. An example of a suitable technique is density gradient centrifugation, for example, using Histopaque 1077 (Sigma Chemical Co.). After centrifugation, cells may be collected and washed twice in phosphate-buffered saline (pH 7.0). The cells may then be re-suspended in a suitable cell culture medium. The selection of a suitable cell culture medium for a given type of cell is routine in the art. When the cells are lymphocytes, a suitable medium may be Karyomax (Life Technologies Inc.). The cells may be re-suspended at a concentration suitable for subsequent analysis, for example, at a concentration of from about 1×10⁶ cells per ml to about 1.5×10⁶ cells per ml before use.

Cell Culture Media

The present invention provides a cell culture medium for inducing premature chromosome condensation in a test cell. Any suitable cell culture medium may be supplemented with one or more mitosis enhancing factors to be used as a cell culture medium of the invention. A suitable cell culture medium is one in which the cell of interest may be maintained in a viable state throughout the duration the induction of premature chromosome condensation. Optionally, the suitable cell culture medium may be one in which the test cell may be maintained for a protracted period of time.

The cell culture media of the present invention will typically comprise various ingredients selected to maintain the viability of the test cells. Such ingredients include, but are not limited to, amino acids, vitamins inorganic salts, buffers or buffer salts, sugars, lipids, trace elements, cytokines and hormones. Suitable cell culture media are commercially available from, for example, Life Technologies Inc.

In preferred embodiments, a cell culture medium of the present invention will comprise one or more mitosis enhancing factors. Mitosis enhancing factors are agents associated with the progression of the cell cycle into mitosis. Mitosis enhancing factors include, but are not limited to, cyclins, cyclin kinases, histone kinases, topoisomerases, SMC proteins, cdk1 substrate, histones, and components of mitosis promoting factor (MPF). In preferred embodiments, the mitosis enhancing factor may be a purified mitosis enhancing factor. The mitosis enhancing factor may be purified to any desired level of purity. Preferably, the mitosis enhancing factor will at least 50% pure, i.e., the mitosis enhancing factor will make up at least 50% by weight of a mitosis-enhancing-factor-containing material to be added to a culture medium. In other preferred embodiments, a mitosis enhancing factor may be 75% or greater pure, 80% or greater pure, 85% or greater pure, 90% or greater pure or 95% or greater pure. In a preferred embodiment, a cell culture medium of the present invention may comprise p34^{cdc2}/cyclin B kinase. Suitable p34^{cdc2}/cyclin B kinase is commercially available from, for example, New England Biolabs.

The mitosis enhancing factor may be added to the medium alone or in combination with other factors. The mitosis enhancing factor may be in the form of a native protein or a mutagenized protein. For example, fusion proteins comprising a mitosis enhancing factor may be used. A mitosis enhancing factor may be placed in frame with a protein or peptide portion of a different protein to produce a fusion protein. The construction of fusion proteins is routine in the art (see, for example, Sambrook et al. (1989) Molecular Cloning: A Laboratory Manual, Cold Spring Harbor Press). In preferred embodiments, the fusion proteins of the present invention may comprise, in addition to a mitosis enhancing factor, one or more ligands for a receptor to facilitate cellular uptake of the fusion protein, nuclear localization signals, purification tags, epitopes or the like. In a preferred embodiment, a cell culture medium of the present invention may comprise a fusion protein comprising a mitosis enhancing factor and a nuclear localization sequence. Suitable nuclear localization signals are known in the art and may be found, for example, in U.S. Pat. Nos. 6,051,429 and 5,736,392.

In addition to mitosis enhancing factors, a cell culture medium of the present invention may comprise one or more energy sources including, but not limited to, ATP and GTP.

A cell culture medium of the present invention may optionally comprise one or more transfection reagents. As used herein, transfection reagent is seen to include any reagent which, when added to a cell culture medium, enhances the uptake by a test cell of a mitosis enhancing factor. Transfection reagents include, but are not limited to, neutral lipids, cationic lipids, mixtures of neutral and cationic lipids, proteins, peptides, lipoproteins, lipopeptides and the like. Suitable transfection reagents may be obtained commercially from, for example, Promega Inc. and Life Technologies Inc. In some preferred embodiments, the transfection reagents of the present invention may comprise a peptide that enhances receptor mediated endocytosis. Examples of such transfection reagents may be found in U.S. Pat. No. 6,103,529. The transfection reagent may be added directly to the media or may be combined with the mitosis enhancing factor prior to the addition of the mitosis enhancing factor to the medium.

A cell culture medium of the present invention may optionally comprise one or more phosphatase inhibitors. In some preferred embodiments, the protein phosphatases may specifically inhibit serine/threonine protein phosphatases. In some preferred embodiments the phosphatase inhibitors may specifically inhibit the protein phosphatases 1 and 2A. Suitable protein phosphatases include, but are not limited to, okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR.

Cell Culture Compositions

The cell culture media of the present invention may be used to formulate cell culture compositions comprising a cell or cell population and a culture medium of the invention. The cell may be any cell in which it is desired to induced premature chromosome condensation. Cells isolated from subjects are particularly preferred. The isolated cells may be derived from any organ or tissue in the subject including, but not limited to, blood, heart, lung, epithelial tissue and/or intestinal tissue.

Kits

The present invention contemplates kits adapted for use in cytogenetic research. Typically, the kits of the invention may comprise one or more containers holding a cell culture medium of the present invention. The cell culture medium may be in liquid form or in the form of a dry powder concentrate. The kits of the invention may comprise one or more containers holding one or more mitosis enhancing factors. The factors may be in solution or may be in the form of a dried powder. Kits of the invention may comprise one or more containers holding one or more phosphatase inhibitors. Optionally, kits of the invention may comprise one or more containers holding one or more transfection reagents and/or one or more energy sources which may be in solution or in dry form.

Kits of the present invention preferably comprise instructions for inducing premature chromosome condensation using the materials and methods of the present invention. In particular, the instructions may provide detailed protocols for inducing premature chromosome condensation in a cell or cell population without the need to stimulate the cell or cell population with a mitogen.

Preparation and Analysis of Chromosome Spreads

PCC spreads may be prepared according to standard cytogenetic procedures directly after the indicated treatment. Briefly, cells may be treated with a hypotonic potassium chloride (0.075 M) solution for 5 minutes and fixed in acetic: methanol (1:3) fixative. Fixed cells may be dropped onto acid cleaned glass slides.

To directly visualize the spread chromosomes, the slides may be stained. Suitable stains are known to those of skill in the art, for example, a 4% aqueous solution of Giemsa stain may be used for observation under a light microscope. Coded slides can be analyzed under 1000×magnification. Cells with condensed chromatin material displaying at least partial separation of chromosomes are scored as PCC spreads.

The PCC index may be determined as follows.

$$\frac{PCC spreads number}{( interphase cell number + PCC spreads number )} \times 100$$

For experiments involving fluorescent in situ hybridization analysis (FISH), after preparing a chromosome spread, whole-chromosome DNA hybridization probe specific for one or more chromosomes. Optionally, a whole chromosome DNA hybridization probe may be directly labeled with a detectable moiety and may be used to analyze the spread chromosomes. Such labeled chromosome-probes are commercially available. As an example, whole chromosome probe specific for chromosome 1 labeled with spectrum green fluorochrome may be obtained from Vysis Inc.

In situ hybridization and chromosome painting may be done using techniques well known in the art (see, for example, Brown et al. (1992) Int. J. Radiat. Oncol. Biol. Phys. 24, 279-286).

In the working example of the invention disclosed below, a chromosome 1 probe from Vysis was used according to the manufacturer's protocol. Other suitable probes are known to those skilled in the art and may be used without departing from the spirit of the invention. Other preferred probes include probes specific for pathological conditions.

Cells may be mounted in a medium containing 4,6-diamidino-2-phenyl-indole (DAPI) for analyzing chromosome 1 aberrations under a fluorescence microscope (Leitz) equipped with filters for DAPI and fluorescein isothiocyanate (FITC).

The coded slides may be observed at a 1000×magnification for analyzing aberrations involving chromosome 1. Chromosome aberration analysis is based on the following general criteria:

The cells to be included in the analysis should show one or more (and preferably all) of the following: (a) at least partial separation of chromosomes with condensed chromatin material as determined by DAPI counterstain, (b) two or more clearly separated chromosome 1-specific spots with bright green fluorescent signals (cells with single green spots, arising because of overlapping signals, were not included), (c) spots that were similar in fluorescent intensity, and (d) an area representing about 15 to 100% of the area of the spots observed in samples from sham-treated controls.

The area of spots in the control samples may not always be uniform because of differential chromosome condensation and, in a few cases, angular presentation under the microscope. In such cases of ambiguity, cells should be excluded from analysis.

It will be readily apparent to those of skill in the art that other suitable modifications and adaptations may be made to the materials and methods of the present invention without departing from the scope of the invention or any embodiment thereof. Having now described the invention in detail, the invention may be more clearly understood with reference to the figures and the following non-limiting examples.

### EXAMPLES

### Example 1

Induction of Premature Chromosome Condensation in Mitogen Stimulated Cells

For purposes of comparison and in order to determine a suitable level of phosphatase inhibitor, premature chromosome condensation was induced in HPBLs using prior art methodology.

HPBLs prepared as described above may be incubated in cell culture medium supplemented with an energy source. In order to determine the optimal OA concentration and duration of incubation for PCC, phytohemagglutinin (PHA, 10 μg/ml; Murex Diagnostics) was subsequently added to the medium to stimulate proliferation. This complete medium did not contain a mitosis enhancing factor.

Incubation of unstimulated HPBL in a cell culture medium containing OA alone did not result in PCC induction, thus, PHA was used to help activate cell cycle progression. The HPBL were treated with OA at concentrations ranging from 0.25 to 1 μM in a cell culture medium containing 100 μM ATP and incubated at 37° C. for varying durations of up to 24 hours. Slides were prepared and PCC index was determined as explained above.

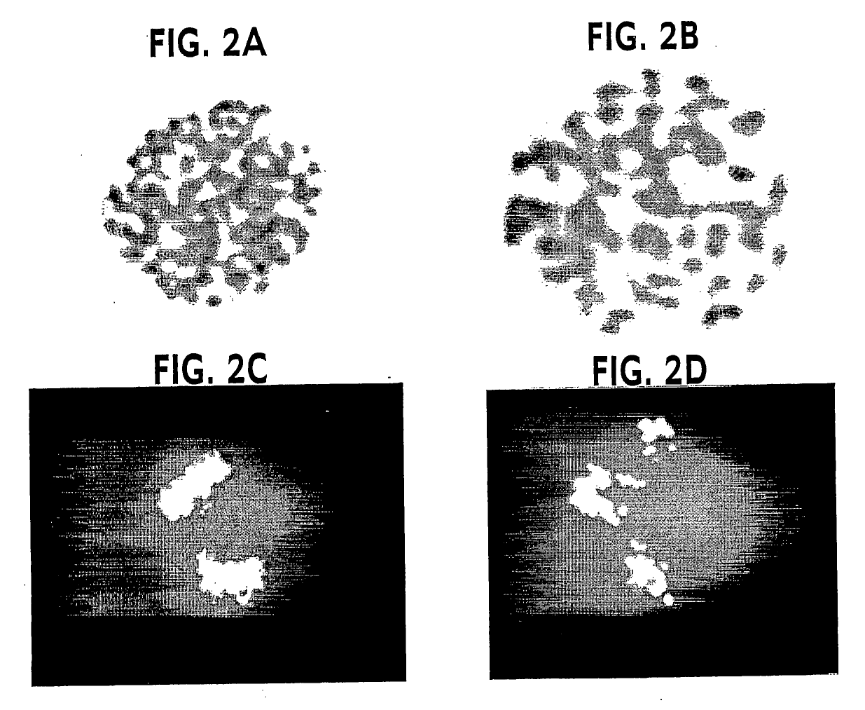

FIG. 2A is a representative photomicrograph showing PCC induced by a treatment with OA in a mitogen-stimulated HPBL stained with Giemsa. Dissolution of cell membrane, condensation of the chromatin material, and partial separation of chromosomes characterized OA-induced PCC. Undivided chromosomes appear less condensed compared to metaphase chromosomes or PCC induced by mitotic-cell fusion technique, and chromosome clumps are still visible in most cells.

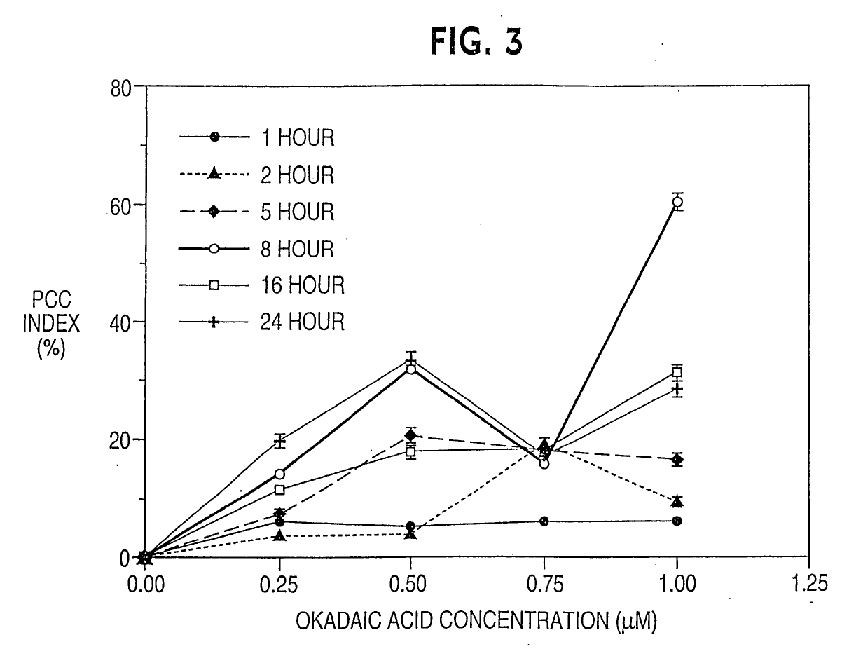

FIG. 3 shows the effect of OA concentration and duration of incubation on PCC induction in the mitogen-stimulated HPBL model. Pooled data is shown from two or more independent experiments with each concentration and time point representing more than 1,000 cells. Treatment of mitogen-stimulated HPBL with OA (0.25 μM) resulted in significant (p<0.01, Student's t-test) PCC levels determined by PCC index within 1 hour, compared with controls. The PCC index reached a maximum of 61% at a 1 μM concentration at 8 hours. At a 0.75-μM concentration, the index peaked at 2 hours, exhibiting PCC in about 20% of cells, and remained at that level for up to 24 hours. It appears that OA at 0.75 μM concentration is not cytostatic and induces a reasonably high PCC yield in mitogen-stimulated HPBL model. Therefore, this concentration was used in further studies with p34^{cdc2}/cyclin B kinase to induce PCC in unstimulated HPBL.

It has been previously demonstrated that treatment of mitogen-stimulated HPBL with phosphatase inhibitors, such as OA or calyculin A, induces premature condensation of chromatin material. In those studies, HPBL were treated, 41 to 45 hours after PHA stimulation, with OA doses between 0.1 and 0.5 μM (Gotoh et al. (1996) Int. J. Radiat. Biol. 70, 517-520; Kanda et al. (1999) Int. J. Radiat. Biol. 75, 441-446) or with 0.05 μM calyculin A (Durante et al. (1998) Int. J. Radiat. Biol. 74, 457-462) for varying durations of 1 to 6 hours to induce entry into a mitosis-like state from the S- or G₂-phase of the cell cycle. In the present experiment, the effects of OA concentrations between 0.25 and 1μM, treated immediately and up to 24 hours after mitogen stimulation of HPBL were studied. PHA was used to help activate cell cycle progression. In this study, significant (p<0.01) elevation in PCC yield was observed as early as 1 hour, indicating PCC induction before DNA replication in a rapidly dedifferentiating cohort of mitogen-stimulated HPBL population. The significant (p<0.01) elevation in PCC index that was observed as early as 1 hour after treatment with OA is comparable to that seen in proliferating cells by others (Gotoh et al. (1996) Int. J. Radiat. Biol. 70, 517-520; Durante et al. (1998) Int. J. Radiat. Biol. 74, 457-462; Coco-Martin et al. (1997) Int. J. Radiat. Biol. 71, 265-273; and Ghosh et al. (1992) Exp. Cell Res. 201, 535-540).

In the optimization study (FIG. 3), 0.75 μM OA resulted in a peak PCC level of 20% at 2 hours and remained at that level for up to 24 hours. This dose was used for treatment with p34^{cdc2}/cyclin B kinase to induce PCC in the unstimulated HPBL model. Selection of this dose was based not only on PCC yield but also on quality of PCC spreads. Similar to the observations of Kanda et al. (Kanda et al. (1999) Int. J. Radiat. Biol. 75, 441-446), prolonged treatment with higher concentrations of OA was observed to result in poor spread quality, possibly due to toxicity. In addition, OA was found to arrest cell cycle progression in human myeloid leukemic cell lines in a concentration- and time-dependent manner (Ishida et al. (1992) J. Cell. Physiol. 150, 484-492). At higher PCC inducible concentrations (above 0.5 μM), cell-cycle arrest occurred at G₁-S-phase; but in lower concentrations cell-cycle arrest occurred at G₂-M phase (Ishida et al. (1992) J. Cell. Physiol. 150, 484-492).

### EXAMPLE 2

Induction of Premature Chromosome Condensation in Resting Cells

In the following working example of the present invention, PCC induction in unstimulated HPBL was accomplished by the addition of p34^{cdc2}/cyclin B kinase to the complete media supplemented with ATP (100 μM) containing OA (0.75 μM) and incubation for three hours at 37° C. PCC index was determined from two or more independent experiments, each data point representing more than 1,000 cells. The pooled data were compared with the yield obtained by OA treatment alone in the mitogen-stimulated HPBL model. The results obtained were compare to the results obtained using the prior art methodology of the preceding example.

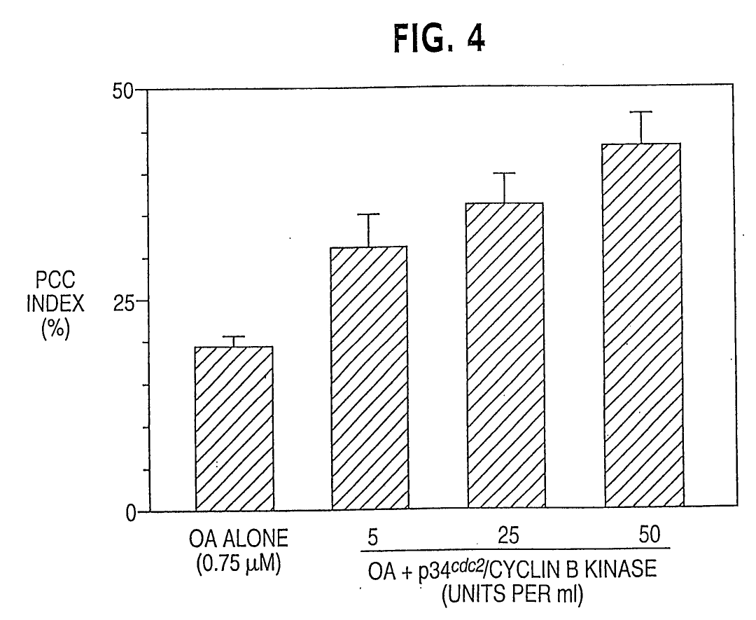

The presence of p34^{cdc2}/cyclin B kinase at concentrations as low as 5 units per ml resulted in PCC induction in unstimulated HPBL. At this concentration, the PCC yield was approximately 30% higher than the yield in the group treated with OA alone in mitogen-stimulated HPBL (FIG. 4). An increase in the enzyme concentration resulted in a concentration-dependent and significant (p<0.05; Student's t-test) increase in PCC yield (FIG. 4). It also improved the spreading and condensation of the chromatin material, yielding better quality PCC spreads (FIG. 2B).

### EXAMPLE 3

Determination of Radiation Dosage Using Chromosome Spreads From Unstimulated Cells

The PCC spreads prepared from unstimulated cells were suitable for detecting radiation-induced chromosome aberrations involving a specific chromosome after hybridization with whole-chromosome probes by the “spot assay” described by Coco-Martin and Begg (Coco-Martin et al. (1997) Int. J. Radiat. Biol. 71, 265-273).

Cell suspension in Karyomax was placed in 15-ml polypropylene centrifuge tubes and, at room temperature, was exposed to gamma rays at a dose rate of 1 Gy/min in a bilateral field of a ⁶⁰Co facility. Radiation source and dosimetry procedures were previously described (Stankus et al. (1995) Int. J. Radiat. Biol. 68, 1-9). The dose rate was measured with a tissue-equivalent ionization chamber before irradiation. The field was uniform within 2%. In radiation dose-response studies, unstimulated HPBL were incubated at 37° C. for 21 hours after exposure in complete medium before PCC induction.

FISH was used to quantify cells with radiation-induced structural aberrations involving chromosome 1 in PCC spreads obtained by incubating unstimulated HPBL in a medium containing OA, ATP, and p34^{cdc2}/cyclin B kinase. The study evaluated the potential application of this “spot assay” to biological dosimetry and included a 24 hour repair incubation at 37° C. following exposure to gamma-ray doses of 0 to 7.5 Gy. PCC spreads were prepared and FISH technique was applied as explained above. Since the maximum difference between experiments was not significant (chi-square value=0.265, p=0.606 for one degree freedom), the data were pooled from four independent experiments, with each dose level representing two or more experiments. At least 1,000 cells were analyzed for enumerating aberrations involving chromosome 1.

In cells that had not been irradiated, two fluorescent (green) spots were seen, reflecting two copies of chromosome 1 (FIG. 2C). Irradiated cells often exhibited more than two fluorescent spots (FIG. 2D) due to induction of aberrations in chromosome 1, which likely reflect radiation-induced fragments or exchanges. The data on frequency distribution of cells with aberrations involving chromosome 1, after exposure to different doses of gamma radiation, are presented in Table 1.

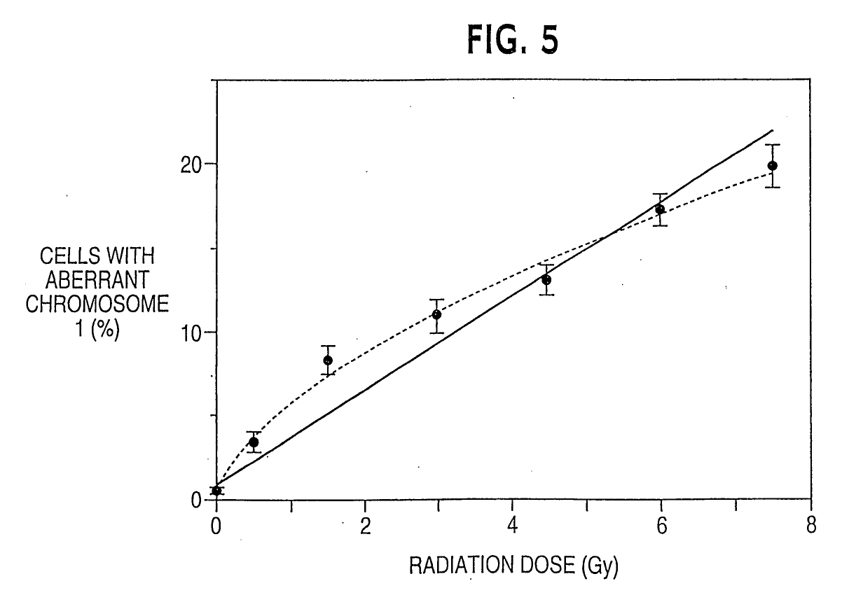

These data demonstrate that the number of cells with aberrant chromosome 1 increases with radiation doses between 0 and 7.5 Gy. This, in general, is in good agreement with dose-effect increase for cytogenetic endpoints. The number of chromosome 1 excess spots increased with radiation dose from 0.035±0.0058 per cell at 0.5 Gy to 0.236±0.0126 at 7.5 Gy. Base-line frequency of cells with chromosome 1 aberrations in FISH-painted PCC spreads was 0.006±0.0020. Frequency of cells with two spots decreased from 0.965 at 0.5 Gy to 0.803 at 7.5 Gy with a corresponding increase in the frequency of cells with more than two spots (Table). The number of cells with more than two spots for chromosome 1 increased with radiation dose from 0 to 7.5 Gy and reached a maximum of 19.70±1.258 per cent (FIG. 5).

The dose-response data for the number of cells with aberrant chromosome 1 were fitted with two models, a linear model (Y=(2.77±0.230) D+0.90±0.431 and r²=0.966) fitted by the weighted least-squares regression method (weights were based on the reciprocal of the SE of the mean squared) and a nonlinear power model (Y=(5.70±0.46)D^{(0.61±0.05)}

TABLE 1
Frequency distribution analysis of cells with aberrant chromosome 1
after exposure to different doses of gamma rays as visualized in PCC
induced by a treatment with okadaic acid and p34^{cdc2} cyclin B kinase^{a}

| | | Frequency of cells | | | | |
|---|---|---|---|---|---|---|
| Radiation | Total | with number of | | | Frequency of cells | Number of excess |
| dose | number | chromosome 1 spots | | | with >2 spots | spots/cell |
| (Gy) | of cells | 2 spots | 3 spots | 4 spots | Mean ± SE | Mean ± SE |
| 0 | 1500 | 0.994 | 0.006 | — | 0.006 ± 0.0020 | 0.006 ± 0.0020 |
| 0.5 | 1000 | 0.965 | 0.035 | — | 0.035 ± 0.0058 | 0.035 ± 0.0058 |
| 1.5 | 1000 | 0.917 | 0.083 | — | 0.083 ± 0.0087 | 0.083 ± 0.0087 |
| 3.0 | 1003 | 0.890 | 0.1096 | — | 0.110 ± 0.0099 | 0.110 ± 0.0099 |
| 4.5 | 1486 | 0.869 | 0.1232 | 0.008 | 0.131 ± 0.8760 | 0.139 ± 0.0088 |
| 6.0 | 1666 | 0.828 | 0.1477 | 0.0240 | 0.172 ± 0.0092 | 0.196 ± 0.0092 |
| 7.5 | 1000 | 0.803 | 0.1580 | 0.0390 | 0.197 ± 0.0126 | 0.236 ± 0.0126 |

^{a}Data were pooled from four independent experiments with each dose level representing two or more experiments.

and r²=0.9901). When fitted with a nonlinear power model, a bending of the dose-response curve towards the abscissa was observed.

The dose-response relationship has a broader dose range than other metaphase-spread based cytogenetic assays or micronucleus assay. With the nonlinear power model fit, the observed downward curvature of the dose-response curve towards the abscissa. Since only one chromosome pair was painted, which represents only a fraction of the genome, some saturation of the signal was expected with increasing radiation dose. This effect is particularly true at higher radiation doses where the number of separate signals produced by complexes (both exchanges and fragments) is restricted, with nuclear area being constant for a given cell. In addition, mean exchanges per cell are known to increase with a positive upward curvature with low-LET radiation. In this case, this curvature was somewhat mitigated because of the inclusion of fragments (which have different dose- response curves) that distorted the curve. The better fit with a nonlinear power model suggests that this assay may be more sensitive at lower radiation doses. This data is in good agreement with earlier data of Coco-Martin and Begg (Coco-Martin et al. (1997) Int. J. Radiat. Biol. 71, 265-273), which involved a measurement of chromosome 4 aberrations induced by gamma irradiation in a human adenocarcinoma cell line (A549) in G₁-phase PCC induced by OA.

### EXAMPLE 4

In Vivo Validation of Determination of Radiation Dosage

The methods disclosed herein can be used to assess the dose of radiation received by a subject. This was demonstrated using premature chromosome condensation spreads of HPBLs performed after a 24 hour repair incubation at 37° C. following exposure to different doses of gamma rays. A base-line frequency of 0.006±0.0020 per cell involving chromosome 1 aberrations was observed in unstimulated HPBL for this assay. This is higher than base-line frequencies for other cytogenetic assays (e.g., dicentrics (0.001 per cell) measured in metaphase spreads). A higher base-line frequency, in general, suggests that some cells carrying aberrations are lost from the cell population before mitosis and, therefore, are not detected by the metaphase-spread-based cytogenetic assays. Thus, the present methods more accurately assess the condition of the cells, since cells that are not competent to undergo mitosis are still represented in the data set and are not lost.

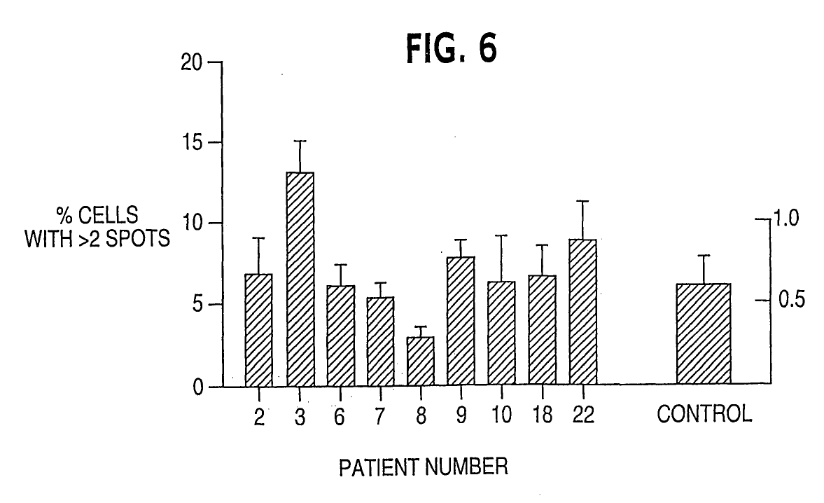

HPBL samples were collected from individuals who had been exposed to ⁶⁰Co gamma radiation from a scrap metal source, a radiation leak occurring in Bankok, Thailand. These individuals received radiation doses of 0.1 to 16 Gy, at a dosage rate of up to 200 μSv/h. From those exposed to the radiation (over 30 people), twelve samples were collected approximately four months after exposure, and nine samples with controls were analyzed by the FISH method described above to determine the number of chromosomal aberrations in chromosome 1. These data are presented in FIG. 6, which shows the increase in the percentage of cells with two or more fluorescent spots in cells isolated from patients exposed to radiation when compared to normal control cells.

The methods of the present invention, as exemplified by the PCC assay performed on HPBL of exposed individuals, provide a direct and sensitive cytogenetic tool for biodosimetry (Pantelias et al. (1985) Mutat. Res. 149, 67-72; Prasanna et al. (1997) Health Phys. 72, 594-600; and Cornforth et al. (1983) Science 222, 1141-1143). The assay can rapidly predict absorbed dose (within 24 hours of the receipt of a blood sample in the laboratory) to enable effective clinical treatment. Since it is conducted on unstimulated cells and does not require cell division, confounding factors such as radiation-induced cell-cycle delay (Poncelet et al. (1988) Strehlanther. und Onkol. 164, 542-543) and death (MacVittie et al. (1996) Advances in the Treatment of Radiation Injury, Elsevier Science, 263-269) do not interfere with dose estimates.

These results indicate that the present method provides a simpler and more reliable techniques for biological dosimetry of radiation exposures than currently used techniques such as analysis of chromosome aberrations in metaphase or PCC spreads after mitotic-cell fusion. The present method involves inducing PCC in unstimulated cells and analyzing aberrations involving specific chromosomes. This method, involving a simple incubation of test cells in a cell culture medium containing a mitosis enhancing factor and optionally a phosphatase inhibitor and an energy source (for example, p34^{cdc2}/cyclin B kinase, OA and ATP), to induce premature chromosome condensation, is simple and does not require the high degree of technical expertise associated with alternative PCC-inducing protocols (Pantelias et al. (1983) Somatic Cell Genet. 9, 533-547; Johnson et al. (1970) Nature 226, 717-722).

### EXAMPLE 5

Examination of Chromosomal Integrity in Oocytes Blastocysts, Stem Cells and Embryonic Cells

Using the methods in Example 2, PCC is induced in a single cell, such as an oocyte, polar body or cell from a blastocyst, or multiple cells, such as an amniotic fluid sample or cells from an established human stem cell line. Oocytes or embryonic cells from mice can also be used. The cell or cells are incubated in the complete medium described in Example 2 for 3 hours at 37° C. Chromosome spreads are prepared, and the chromosomes are examined using any of the methods described on page 15. Structural abnormalities are indicated, e.g., by more than 2 bright fluorescent spots, using the FISH technique, or by failure of a locus specific probe to bind to a chromosome. Healthy embryos or cell lines are maintained in culture or in utero, and healthy oocytes, whose corresponding polar bodies are tested, are fertilized. Abnormal cells are not maintained in culture or used in further procedures.

For optimization of the complete medium, samples containing multiple cells can be split into portions, each of which is incubated in the complete medium of Example 2, but in which each portion contains a different concentration of a phosphatase inhibitor (okadaic acid or calyculin A) or an energy source (ATP) or a cyclin kinase (p34^{cdc2}/cyclin B kinase). Multiple components can be optimized, according to the number of sample portions available. After three hours at 37° C., the cells are harvested, subjected to hypotonic treatment, fixed with methanol/acetic acid, placed on slides and stained to obtain chromosome spreads. The percentage of cells in which PCC is induced is calculated for each sample, and a dose-response relationship is determined. The optimal concentration of one or more components is then used to prepare complete medium for subsequent analyses.

Micromanipulation techniques are used to manipulate single-cell embryos or oocytes. The cell is held attached to a micropipette tip and contained in a culture dish with complete medium. The cell is incubated in medium for several hours at 37° C. prior to induction of PCC. Alternatively, a solution of p34^{cdc2}/cyclin B kinase and either okadaic acid or calyculin A is introduced into the cell by microinjection or by electroporation. The contents of the dish are then replaced with, successively, hypotonic solution and fixative, and a chromosome spread is prepared. As a second alternative, the cell is held within a capillary tube containing complete medium for incubation, and the aforementioned treatments performed by aspiration and refilling. This procedure is carried out under a stereomicroscope. A chromosome spread is prepared in a similar fashion.

The chromosomes are examined by in situ hybridization, chromosome painting or fluorescence microscopy, as described above. Whole-chromosome DNA hybridization, in which the chromosome is labeled with a commercially available fluorochrome, is specific for single chromosomes. In situ hybridization and chromosome painting are carried out according to standard methods. Following PCC induction, the cell sample is mounted in medium containing DAPI under a fluorescence microscope equipped with filters for DAPI and FITC. Chromosome aberrations, such as those studied in chromosome 1, are visible and can be analyzed for type and number.

### EXAMPLE 6

High-throughput Isolation of PCC-sensitive Lymphocyte Subpopulations

For cytogenetic applications and analyses involving large numbers of samples, high-throughput procedures for isolating subpopulations of lymphocytes that are susceptible to PCC are required. Currently procedures are tedious and inefficient, i.e., isolation on a density gradient (e.g., Ficoll, Histopaque), followed by treatment with a mitogen and PHA. Metaphase spreads are then prepared, and the cycle arrested by treatment with colcemid. These cells are then cultured, all to produce a subpopulation with a mitotic yield of 4-5%.

To quickly and simply produce adequate numbers of PCC-sensitive peripheral blood lymphocytes, whole blood is mixed with a cocktail containing RosetteSep® (Stem Cell Technologies) multivalent antibodies in centrifuge tubes, e.g., 50 ml conical centrifuge tubes. The tubes are incubated for 20 minutes at room temperature. Mitogen and PCC-insensitive lymphocytes and non-lymphocytic white blood cells are cross-linked by the antibodies to form tetrameric “rosette” complexes. The contents in each tube are then underlaid with Ficoll, and the tubes are spun for 20 minutes. An interface containing a purified lymphocyte subpopulation that is PCC-sensitive is formed between an upper plasma layer and a lower Ficoll layer. Unwanted white blood cells, red cells and other cellular and particulate blood components are pelleted to the bottom.

This procedure is scalable to include a large number of blood samples (>500 per run using an automated isolation system), and a ten-fold increase mitotic yield is achievable. As a result, this procedure is preferable to current methods for cytogenetic applications. For clinical applications related to immune system disorders, this procedure is well-suited for the isolation of T cell subpopulations such as CD3+ T cells, CD4+ T cells and CD8+ T cells.

Isolation of PCC-sensitive lymphocyte subpopulations is also accomplished using StemSep® (Stem Cell Technologies) immunomagnetic cell selection assay. In this assay, the reagent cocktails consist of antibodies directed against markers present on the surface of the unwanted cells in the sample. The cells labeled by these antibodies are efficiently removed by passage through a magnetic column, while the desired cells are collected in the column flow through, unlabeled and highly enriched. StemSep® immunomagnetic negative cell selection is used for isolation of memory CD4+ T cells (CD4+ T cell cocktail plus CD45 RA), Resting CD4+ T cell (CD4+ T cell cocktail plus one or more of CD25, CD69, HLA-DR), Resting CD8+ T cell (CD8+ T cell cocktail plus one or more of CD25, CD27, CD69, HLA-DR), αβ T cell (T cell cocktail plus TCRγδ) and γδ T cell (T cell cocktail plus TCR γδ).

Having fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof. Any such modifications or changes are intended to be within the scope of the appended claims.

All publications, patents and patent applications mentioned in this specification are indicative of the level of skill of those skilled in the art to which this invention pertains and are specifically incorporated herein by reference.

What is claimed:

1. A culture medium for inducing premature chromosome condensation in a cell comprising a cyclin kinase having mitosis enhancing properties, wherein the cyclin kinase is present in an amount effective to induce premature chromosome condensation.

2. A medium according to claim 1, wherein the cyclin kinase is p34^{cdc2}/cyclin B kinase.

3. A medium according to claim 1, further comprising a phosphatase inhibitor.

4. A medium according to claim 3, wherein the phosphatase inhibitor is selected from a group consisting of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR.

5. A medium according to claim 1, further comprising an energy source.

6. A medium according to claim 5, wherein the energy source is selected from a group consisting of ATP and GTP.

7. A medium according to claim 1, further comprising a transfection reagent.

8. A kit comprising the medium of any one of claims 1-7.

9. A method of analyzing a chromosome, comprising:
(a) incubating a cell with a medium comprising a cyclin kinase having mitosis enhancing properties, wherein the cyclin kinase is present in an amount effective to induce premature chromosome condensation; and
(b) analyzing the condensed chromosome.

10. A method of assessing clastogenicity of a compound, comprising:
(a) contacting a cell with the compound;
(b) incubating the cell with a medium comprising a cyclin kinase having mitosis enhancing properties, wherein the cyclin kinase is present in an amount effective to induce premature chromosome condensation; and
(c) analyzing the condensed chromosomes for breakage, structural and/or numerical aberrations.

11. A method according to claim 10, wherein the cell is contacted with the medium and the compound simultaneously.

12. A method according to claim 10, further comprising incubating the cell after contact with the compound for a period of time sufficient to allow chromosomal repair.

13. A method of assessing toxicity of a compound, comprising:
(a) contacting a cell with the compound;
(b) incubating the cell with a medium comprising a cyclin kinase having mitosis enhancing properties, wherein the cyclin kinase is present in an amount effective to induce premature chromosome condensation; and
(c) analyzing the condensed chromosomes.

14. A method according to claim 13, wherein the cell is contacted with the medium and the compound simultaneously.

15. A method according to claim 13, further comprising incubating the cell after contact with the compound for a period of time sufficient to allow chromosomal repair.

16. A method of detecting chromosomal abnormalities in a subject, comprising:
(a) isolating one or more cells from the subject;
(b) contacting at least one cell with a medium comprising a cyclin kinase having mitosis enhancing properties, wherein the cyclin kinase is present in an amount effective to induce premature chromosome condensation; and
(c) analyzing the condensed chromosomes for chromosome abnormalities.

17. A method according to claim 16, wherein chromosomal abnormalities are analyzed based on an evaluation of the number of condensed chromosome domains or spots within a cell.

18. A method according to claim 17, wherein the subject is in utero.

19. A method according to claim 17, wherein the abnormality is a numerical abnormality.

20. A method according to claim 17, wherein the abnormality is a structural abnormality.

21. A method of assessing a radiation dose received by a subject, comprising:
(a) isolating one or more cells from the subject;
(b) contacting at least one cell with a medium comprising a cyclin kinase having mitosis enhancing properties, wherein the cyclin kinase is present in an amount effective to induce premature chromosome condensation; and
(c) analyzing the condensed chromosomes for chromosome abnormalities.

22. A method according to any one of claims 9, 10, 13, 16 or 21, wherein the cyclin kinase is p34^{cdc2}/cyclin B kinase.

23. A method according to any one of claims 9, 10, 13, 16 or 21, wherein the medium further comprises a phosphatase inhibitor.

24. A method according to claim 23, wherein the phosphatase inhibitor is selected from a group consisting of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR.

25. A method according to any one of claims 9, 10, 13, 16 or 21, wherein the medium further comprises an energy source.

26. A method according to claim 25, wherein the energy source is selected from a group consisting of ATP and GTP.

27. A method according to any one of claims 9, 10, 13, 16 or 21, wherein the medium further comprises a transfection reagent.

28. A method according to any one of claims 9, 10, 13, 16 or 21, wherein the cell is a lymphocyte.

29. A method according to any one of claims 9, 10, 13, 16 or 21, wherein the cell is a mammalian cell.

30. A method according to any one of claims 9, 10, 13, 16 or 21, wherein the cell is a human peripheral blood lymphocyte.

31. A method according to any one of claims 9, 10, 13, 16 or 21, wherein the cell is a murine cell.

32. A method according to any one of claims 9, 10, 13, 16 or 21, wherein the cell is a murine peripheral blood lymphocyte.

33. A method according to any one of claims 9, 10, 13, 16 or 21, wherein analyzing the chromosome comprises preparing a chromosome spread.

34. A method according to any one of claims 9, 10, 13, 16 or 21, wherein analyzing the chromosomes comprises hybridizing an oligonucleotide to at least one or more chromosomes and enumerating chromosome spots.

35. A method according to claim 34, wherein the oligonucleotide comprises a detectable moiety.

36. A method according to claim 35, wherein the detectable moiety is a fluorescent moiety.

37. A method according to claim 35, wherein the detectable moiety is selected from a group consisting of biotin, digoxigenin, antigens, enzymes and haptens.

38. A composition comprising a cell and a cell culture medium comprising a cyclin kinase having mitosis enhancing properties, wherein the cyclin kinase is present in an amount effective to induce premature chromosome condensation.

39. A composition according to claim 38, wherein the cyclin kinase is p34^{cdc2}/cyclin B kinase.

40. A composition according to claim 38, further comprising a phosphatase inhibitor.

41. A composition according to claim 40, wherein the phosphatase inhibitor is selected from a group consisting of okadaic acid, salts of okadaic acid, calyculin A, cantharidic acid, cantharidin, cypermethrin, deltamethrin, dephostatin, 3,4-dephostatin, endothall, fenvalerate, fostriecin, microcystin-LA, microcystin-LF, microcystin-LR, microcystin-LW, microcyctin-RR, and microcystin-YR.

42. A composition according to claim 38, further comprising an energy source.

43. A composition according to claim 42, wherein the energy source is selected from a group consisting of ATP and GTP.

44. A composition according to claim 38, further comprising a transfection reagent.

45. A kit comprising the composition of any one of claims 38-44.
